(19) United States
(12) Patent Application Publication
(10) Pub. No.: US 2008/0093301 A1
(43) Pub. Date: Apr. 24, 2008

(54) Disubstituted Cucurbituril-Bonded Silica Gel

(75) Inventors: Kimoon Kim, Pohang-city (KR); Dong- Hyun Oh, Pohang-city (KR); Nagarajan Erumaipatty Rajagounder, Pohang-city (KR); Nandha Kumar Raju, Pohang-city (KR); Ju Young Choi, Pohang-city (KR); Young Ho Ko, Pohang-city (KR)

Correspondence Address: ROTHWELL, FIGG, ERNST & MANBECK, P.C., 1425 K STREET, N.W., SUITE 800, WASHINGTON, DC, 20005, US

(73) Assignee: Postech Academy-Industry Foundation, Pohang-city, Kyungsangbuk-do (KR)

(21) Appl. No.: 11/587,047

(22) PCT Filed: Apr. 20, 2005
(86) PCT No.: PCT/KR05/01127

§ 371 Date: Oct. 20, 2006

(30) Foreign Application Priority Data
Apr. 20, 2004 (KR) 10-2004-0027153

(51) Int. Cl. B01D 15/08; B01D 39/00; C07D 487/22

(52) U.S. Cl. 210/656; 210/198.2; 210/502.1; 540/472

(57) ABSTRACT

A disubstituted cucurbituril-bonded silica gel and its use are provided. The disubstituted cucurbituril-bonded silica gel is useful for removal of air pollutants or water contaminants, and separation and purification of biological, organic, inorganic, or ionic substances.

### REFERENCE TO RELATED APPLICATION

This application is a U.S.C. § 371 National Phase Entry Application from PCT/KR005/001127, filed Apr. 20, 2005, and designating the U.S.

### BACKGROUND OF THE INVENTION

1. Field of the Invention

The present invention relates to a disubstituted cucurbituril-bonded silica gel, and more particularly, to a disubstituted cucurbituril-bonded silica gel, a method of preparing a column stationary phase for column chromatography using the same, and a use of the disubstituted cucurbituril-bonded silica gel in removal of air pollutants or water contaminants, and separation and purification of biologically important substances, organic substances, inorganic substances, or ionic substances.

2. Description of the Related Art

Host molecules such as cyclodextrin (U.S. Pat. No. 4,539,399) and crown ether (Korean Patent No. 026382) have retention capacity for various compounds. In this respect, applications of the host molecules in separation and removal of substances have been studied. To use the host molecules as column packing materials, the host molecules must be covalently bonded to solid substrates such as silica gel, zeolite, titanium oxide, cellulose, and polymeric microspheres. The host molecules covalently bonded to the solid substrates are used as stationary phases of various column packing materials in column chromatography for separation of various test samples.

Cucurbiturils are newly emerging host molecules. Unlike cyclodextrin, cucurbiturils can form a non-covalent linkage with guest molecules including various hydrophilic compounds as well as hydrophobic compounds, in particular, amine-substituted biochemical compounds [J. Am. Chem. Soc. 2001, 123, 11316, EP 1094065, J. Org. Chem., 1986, 51, 1440]. When such cucurbiturils are used in preparation of stationary phases for column chromatography, application to separation of various hydrophilic compounds and hydrophobic compounds, in particular, various biochemically important alkylamines, polypeptides, and proteins is anticipated.

However, cucurbiturils known hitherto have no functional groups for linkage to solid substrates, and thus, utilities of the cucurbiturils as column stationary phases have been limited.

In view of this problem, the present applicant reported hydroxycucurbiturils in which 2n hydroxy groups are introduced in each cucurbit[n]uril molecule (PCT/KR02/02213). However, an efficient linkage of a sufficient number of hydroxycucurbiturils to a silica gel is not ensured. Therefore, development of materials capable of more efficiently bonding cucurbiturils to silica gels is required.

### SUMMARY OF THE INVENTION

The present invention provides a disubstituted cucurbituril-bonded silica gel that has a functional group able to bind with a solid substrate and is useful as a stationary phase for column chromatography and a preparation method thereof, and a silica monolithic column tube using the silica gel and a fabrication method thereof.

The present invention also provides a column packing material and a filter using the disubstituted cucurbituril-bonded silica gel.

The present invention also provides a use of the silica monolithic column tube, the column packing material, and the filter.

According to an aspect of the present invention, there is provided a disubstituted cucurbituril-bonded silica gel in which (i) a disubstituted cucurbituril represented by the following formula 1 is bonded to a modified silica gel represented by the following formula 2 or (ii) a silane compound represented by the following formula 3 bonded to the disubstituted cucurbituril represented by the following formula 1 is bonded to an unmodified silica gel represented by the following formula 2a:

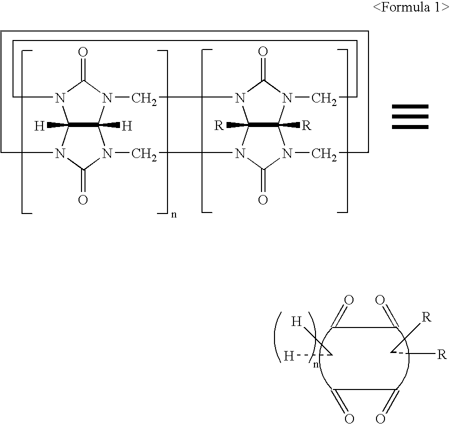

wherein,

n is an integer of 4-7;

Rs are the same or different and each represents a substituted or unsubstituted alkenyl group of C2-C30, a substituted or unsubstituted alkynyl group of C2-C30, a substituted or unsubstituted alkylcarboxyl group of C1-C30, a substituted or unsubstituted hydroxyalkyl group of C1-C30, a substituted or unsubstituted alkoxy group of C1-C30, a substituted or unsubstituted nitroalkyl group of C1-C30, a substituted or unsubstituted aminoalkyl group of C1-C30, a substituted or unsubstituted aryl group of C6-C30, or a substituted or unsubstituted heteroaryl group of C2-C30,

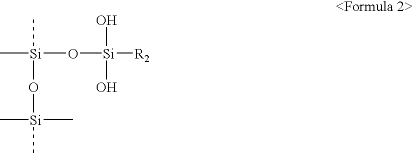

wherein R₂ represents a halogenated alkyl group of C1-C10, a mercaptoalkyl group of C1-C10, an aminoalkyl group of C1-C10, an epoxyalkyloxyalkyl group of C2-C10, an isocyanatoalkyl group of C2-C10, or an isothiocyanatoalkyl group of C2-C10, 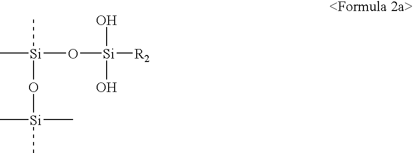
wherein R₂ is a hydroxy group, and
   XSiR′₃ <Formula 3>

wherein X represents a halogenated alkyl group of C1-C10, an aminoalkyl group of C1-C10, an epoxyalkyloxyalkyl group of C2-C10, an isocyanatoalkyl group of C2-C10, or an isothiocyanatoalkyl group of C2-C10; and

R′s are the same or different and each represents a hydrogen, a halogen atom, an allyl group, an alkyl group of C1-C20, a halogenated alkyl group of C1-C20, or an alkyloxy group of C1-C20.

According to another aspect of the present invention, there is provided a disubstituted cucurbituril-covalently bonded silica monolithic column tube obtained by allowing a disubstituted cucurbituril-bonded silane compound obtained by bonding a disubstituted cucurbituril of the following formula 1 to a silane compound of the following formula 3 to pass through a silica monolithic capillary tube:

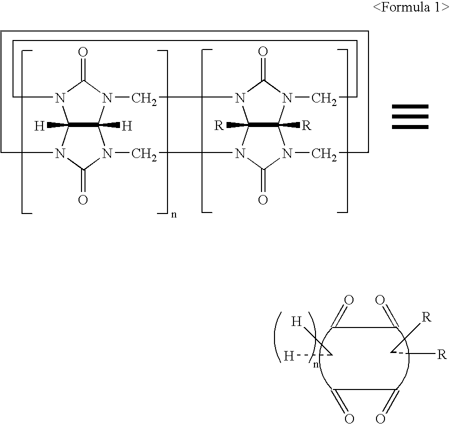

wherein,

n is an integer of 4-7;

Rs are the same or different and each represents a substituted or unsubstituted alkenyl group of C2-C30, a substituted or unsubstituted alkynyl group of C2-C30, a substituted or unsubstituted alkylcarboxyl group of C1-C30, a substituted or unsubstituted hydroxyalkyl group of C1-C30, a substituted or unsubstituted alkoxy group of C1-C30, a substituted or unsubstituted nitroalkyl group of C1-C30, a substituted or unsubstituted aminoalkyl group of C1-C30, a substituted or unsubstituted aryl group of C6-C30, or a substituted or unsubstituted heteroaryl group of C2-C30, and
XSiR′₃ <Formula 3>

wherein X represents a halogenated alkyl group of C1-C10, an aminoalkyl group of C1-C10, an epoxyalkyloxyalkyl group of C2-C10, an isocyanatoalkyl group of C2-C10, or an isothiocyanatoalkyl group of C2-C10; and

R′s are the same or different and each represents a hydrogen, a halogen atom, an allyl group, an alkyl group of C1-C20, a halogenated alkyl group of C1-C20, or an alkyloxy group of C1-C20.

The disubstituted cucurbituril-covalently bonded silica monolithic column tube may be packed with a compound represented by the following formula 4, 6, or 7:

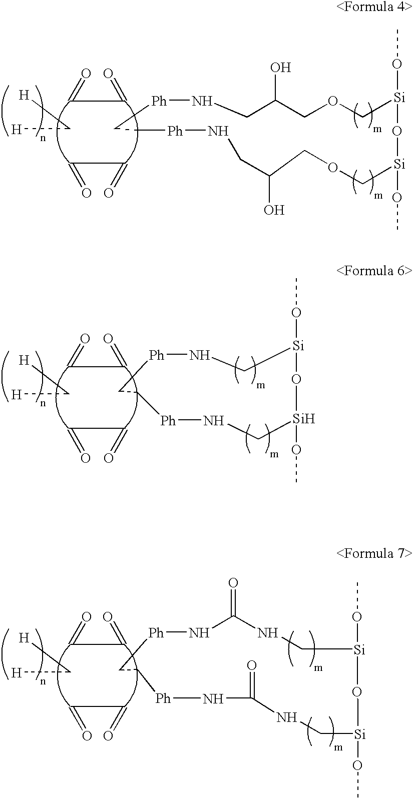

wherein n is an integer of 4-7, m is an integer of 1-10, and -Ph- is phenylene.

According to still another aspect of the present invention, there is provided a method of preparing a disubstituted cucurbituril-bonded silica gel, which includes reacting a disubstituted cucurbituril represented by the following formula 1 with a modified silica gel represented by the following formula 2:

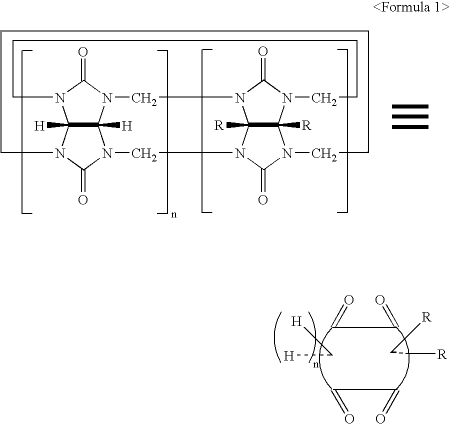

wherein,

n is an integer of 4-7;

Rs are the same or different and each represents a substituted or unsubstituted alkenyl group of C2-C30, a substituted or unsubstituted alkynyl group of C2-C30, a substituted or unsubstituted alkylcarboxyl group of C1-C30, a substituted or unsubstituted hydroxyalkyl group of C1-C30, a substituted or unsubstituted alkoxy group of C1-C30, a substituted or unsubstituted nitroalkyl group of C1-C30, a substituted or unsubstituted aminoalkyl group of C1-C30, a substituted or unsubstituted aryl group of C6-C30, or a substituted or unsubstituted heteroaryl group of C2-C30, and

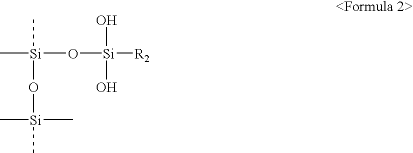

wherein R₂ represents a halogenated alkyl group of C1-C10, a mercaptoalkyl group of C1-C10, an aminoalkyl group of C1-C10, an epoxyalkyloxyalkyl group of C2-C10, an isocyanatoalkyl group of C2-C10, or an isothiocyanatoalkyl group of C2-C10.

According to still another aspect of the present invention, there is provided a method of preparing a disubstituted cucurbituril-bonded silica gel, which includes reacting a disubstituted cucurbituril represented by the following formula 1 with a silane compound represented by the following formula 3 followed by reacting with an unmodified silica gel represented by the following formula 2a:

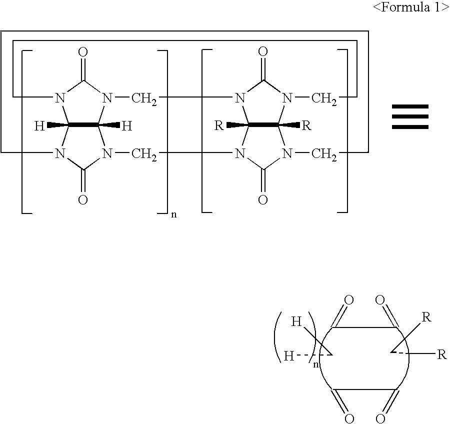

wherein,

n is an integer of 4-7;

Rs are the same or different and each represents a substituted or unsubstituted alkenyl group of C2-C30, a substituted or unsubstituted alkynyl group of C2-C30, a substituted or unsubstituted alkylcarboxyl group of C1-C30, a substituted or unsubstituted hydroxyalkyl group of C1-C30, a substituted or unsubstituted alkoxy group of C1-C30, a substituted or unsubstituted nitroalkyl group of C1-C30, a substituted or unsubstituted aminoalkyl group of C1-C30, a substituted or unsubstituted aryl group of C6-C30, or a substituted or unsubstituted heteroaryl group of C2-C30, 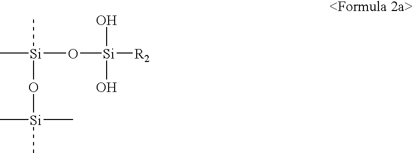
wherein R₂ is a hydroxy group, and
   XSiR′₃ <Formula 3>

wherein X represents a halogenated alkyl group of C1-C10, an aminoalkyl group of C1-C10, an epoxyalkyloxyalkyl group of C2-C10, an isocyanatoalkyl group of C2-C10, or an isothiocyanatoalkyl group of C2-C10; and

R′s are the same or different and each represents a hydrogen, a halogen atom, an allyl group, an alkyl group of C1-C20, a halogenated alkyl group of C1-C20, or an alkyloxy group of C1-C20.

According to still another aspect of the present invention, there is provided a method of fabricating a disubstituted cucurbituril-bonded silica monolithic column tube, which includes: reacting a disubstituted cucurbituril represented by the following formula 1 with a silane compound represented by the following formula 3 to obtain a disubstituted cucurbituril-bonded silane compound, and allowing the disubstituted cucurbituril-bonded silane compound to pass through a silica monolithic capillary tube to obtain a disubstituted cucurbituril-covalently bonded silica monolithic column tube:

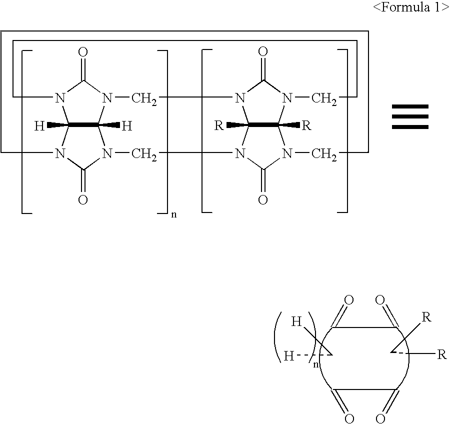

wherein,

n is an integer of 4-7;

Rs are the same or different and each represents a substituted or unsubstituted alkenyl group of C2-C30, a substituted or unsubstituted alkynyl group of C2-C30, a substituted or unsubstituted alkylcarboxyl group of C1-C30, a substituted or unsubstituted hydroxyalkyl group of C1-C30, a substituted or unsubstituted alkoxy group of C1-C30, a substituted or unsubstituted nitroalkyl group of C1-C30, a substituted or unsubstituted aminoalkyl group of C1-C30, a substituted or unsubstituted aryl group of C6-C30, or a substituted or unsubstituted heteroaryl group of C2-C30, and
XSiR′₃ <Formula 3>

wherein X represents a halogenated alkyl group of C1-C10, an aminoalkyl group of C1-C10, an epoxyalkyloxyalkyl group of C2-C10, an isocyanatoalkyl group of C2-C10, or an isothiocyanatoalkyl group of C2-C10; and

R′s are the same or different and each represents a hydrogen, a halogen atom, an aryl group, an alkyl group of C1-C20, a halogenated alkyl group of C1-C20, or an alkyloxy group of C1-C20.

According to still another aspect of the present invention, there is provided a column packing material using the above-described disubstituted cucurbituril-bonded silica gel.

According to still another aspect of the present invention, there is provided a filter using the above-described disubstituted cucurbituril-bonded silica gel.

According to yet another aspect of the present invention, there is provided a use of the above-described silica monolithic column tube in separation of alkylamines, arylamines, polypeptides, or neural substances.

According to a further aspect of the present invention, there is provided a use of the above-described column packing material or filter in separation of hydrophilic amino acids, alkaloids, proteins, nucleic acids, optically or non-optically active asymmetrical substances, drugs, ionic substances, amines, or gaseous compounds.

### BRIEF DESCRIPTION OF THE DRAWINGS

The above and other features and advantages of the present invention will become more apparent by describing in detail exemplary embodiments thereof with reference to the attached drawings in which:

FIG. 1 illustrates a separation result of amino acids according to Example 4 of the present invention.

### DETAILED DESCRIPTION OF THE INVENTION

Hereinafter, the present invention will be described in more detail.

In a disubstituted cucurbituril of the following formula 1 of the present invention, only two functional groups of each cucurbituril molecule are substituted according to the purpose of use so that the disubstituted cucurbituril can be more efficiently bonded to a solid substrate. Therefore, the disubstituted cucurbituril-bonded solid substrate can be used as a stationary phase for column chromatography. In the formula 1, n is 4-7. According to the value n, the disubstituted cucurbituril indicates a disubstituted cucurbit[n+1]uril. That is, the disubstituted cucurbituril of the present invention includes disubstituted cucurbit[5]uril through disubstituted cucurbit[8]uril.

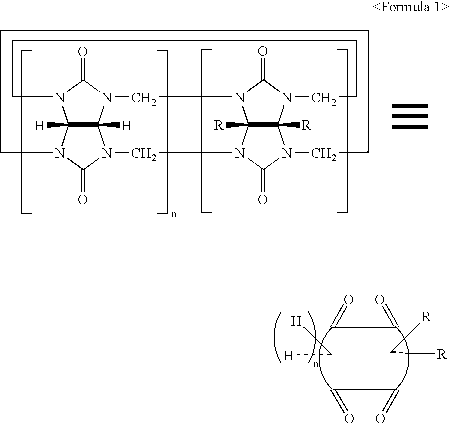

wherein each R is, in particular, selected from the group consisting of a 2-nitrophenyl group, a 3-nitrophenyl group, a 4-nitrophenyl group, a 2-methoxyphenyl group, a 3-methoxyphenyl group, a 4-methoxyphenyl group, a 2-aminophenyl group, a 3-aminophenyl group, a 4-aminophenyl group, a 2-hydroxyphenyl group, a 3-hydroxyphenyl group, and a 4-hydroxyphenyl group.

A synthetic method of the disubstituted cucurbituril of the formula 1 is disclosed in Korean Patent Application No. 2003-6356 filed by the present applicant, the disclosure of which is incorporated herein in its entirety by reference.

In the present invention, the disubstituted cucurbituril of the above formula 1 is covalently bonded to a modified silica gel represented by the following formula 2 having various end functional groups to form a desired compound:

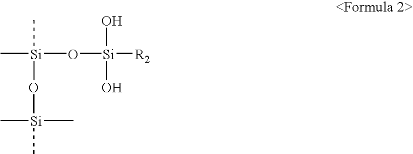

wherein R₂ may be any one of various functional groups according to a desired purpose, and preferably, represents a halogenated alkyl group of C1-C10, a mercaptoalkyl group of C1-C10, an aminoalkyl group of C1-C10, an epoxyalkyloxyalkyl group of C2-C10, an isocyanatoalkyl group of C2-C10, or an isothiocyanatoalkyl group of C2-C10.

Examples of the mercaptoalkyl group with an alkyl moiety of C1-C10 include a 3-mercaptopropyl group and a 5-mercaptopentyl group, examples of the aminoalkyl group of C1-C10 include a 3-aminopropyl group and a 5-aminopentyl group, an example of the epoxyalkyloxyalkyl group with an alkyl moiety of C2-C10 includes a glycidoxypropyl group, examples of the isocyanatoalkyl group with an alkyl moiety of C2-C10 include a 3-isocyanatopropyl group and a 5-isocyanatopentyl group, and an example of the isothiocyanatoalkyl group of C2-C10 includes 3-[3-isothiocyanatophenyl)thioureido]propyl.

The modified silica gel of the formula 2 is a material having a network structure and can be synthesized by a known synthetic method (U.S. Pat. No. 4,539,399; J. Chromatogr. 628 (1993) 11; Tetrahedron Lett. 26 (1985) 3361). For example, the modified silica gel of the formula 2 can be synthesized by reacting a silane having an end functional group such as a mercapto group, an amino group, or an epoxy group, with an uncoated silica gel that is used for column purification.

The disubstituted cucurbituril-bonded silica gel has a covalent linkage between the disubstituted cucurbituril of the formula 1 wherein an end functional group, i.e., each R is a carboxyl group, an amino group, a hydroxy group, or an aryl group, and the modified silica gel of the formula 2 wherein an end functional group, i.e., R₂ is an amino group, an epoxy group, or a thiol group.

The disubstituted cucurbituril-bonded silica gel can be obtained by condensation reaction between diaminophenylcucurbituril of the formula 1 wherein each R is a phenyl group having an amino group at 3 (meta) or 4 (para) position and the modified silica gel of the formula 2.

Examples of the disubstituted cucurbituril-bonded silica gel include compounds represented by the following formulae 4 through 7:

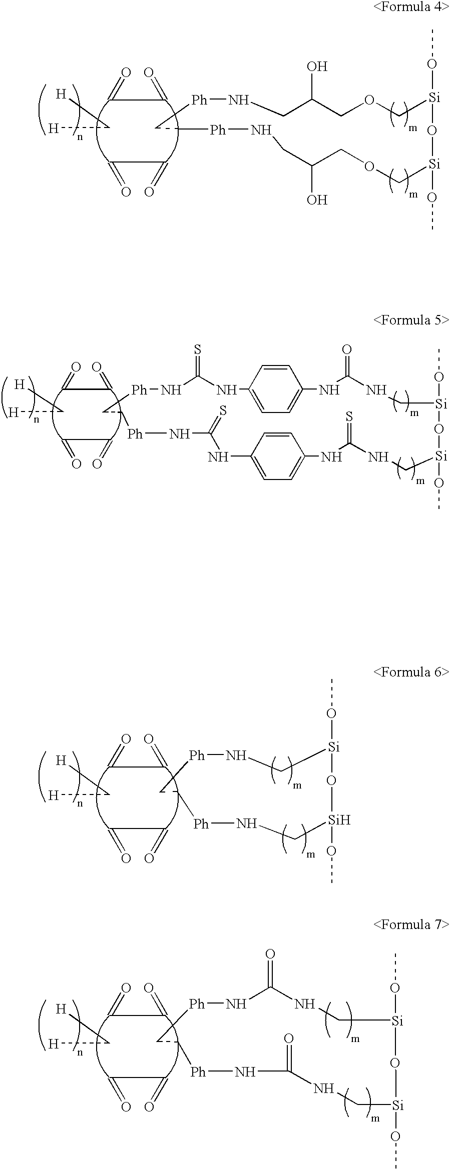

wherein n is an integer of 4-7, m is an integer of 3-10, and -Ph- is phenylene.

The compounds of the formulae 4 through 7 can be prepared as follows.

The compound of the formula 4 can be obtained by condensation reaction between diaminophenylcucurbituril of the formula 1 wherein each R is a phenyl group having an amino substituent at 3 (meta) or 4 (para) position and a modified silica gel of the formula 2 wherein R₂ is a glycidoxyalkyl group with an end epoxy moiety. Non-limiting examples of a solvent used in the condensation reaction include dimethylsulfoxide (DMSO) and dimethylformamide (DMF). The condensation reaction may be carried out in the absence of a base or in the presence of a base such as trimethylamine, potassium carbonate, sodium hydroxide, and pyridine. The reaction temperature varies according to the type of a starting material and the use of the base but may be in a range of 20 to 100° C.

The compounds of the formulae 5, 6, and 7 can be obtained in the same manner as in the preparation of the compound of the formula 4 except that a modified silica gel of the formula 2 wherein R₂ is an isothiocyanatoalkyl group with an end isothiocyanato moiety, a modified silica gel of the formula 2 wherein R₂ is a halogenated alkyl group, and a modified silica gel of the formula 2 wherein R₂ is an isocyanatoalkyl group with an end isocyanato moiety are respectively used instead of the modified silica gel of the formula 2 wherein R₂ is a glycidoxyalkyl group with an end epoxy moiety.

The present invention also provides a silane compound-bonded disubstituted cucurbituril obtained by organic reaction between the disubstituted cucurbituril of the formula 1 and a silane compound represented by the following formula 3. The silane compound-bonded disubstituted cucurbituril is covalently bonded to a silica gel of the following formula 2a to give a disubstituted cucurbituril-bonded silica gel:
XSiR′₃ <Formula 3>

wherein X represents a halogenated alkyl group of C1-C10, an aminoalkyl group of C1-C10, an epoxyalkyloxyalkyl group of C2-C10, an isocyanatoalkyl group of C2-C10, or an isothiocyanatoalkyl group of C2-C10, and R′s are the same or different and each represents a hydrogen, a halogen atom, an aryl group, an alkyl group of C1-C20, a halogenated alkyl group of C1-C20, or an alkyloxy group of C1-C20, and

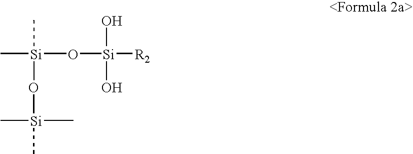

wherein R₂ is a hydroxy group.

Examples of the disubstituted cucurbituril-bonded silica gel include the above-described compounds of the formulae 4, 6, and 7.

In the formula 3, an example of the halogenated alkyl group of C1-C10 includes 3-chlorinated propyltriethoxysilane, examples of the halogen atom include chlorine and bromine, examples of the alkyloxy group include a methoxy group and an ethoxy group, an example of the aminoalkyl group of C1-C10 includes 3-aminopropyltrimethoxysilane, an example of the epoxyalkyloxyalkyl group of C2-C10 includes 3-glycidoxypropyltrimethoxysilane, an example of the isocyanatoalkyl group of C2-C10 includes triethoxysilylpropylisocyanate, and an example of the isothiocyanatoalkyl group of C2-C10 includes 1-(4-isothiocyanatophenyl)-3-trimethoxysilylpropylthiourea.

The silane compound-bonded disubstituted cucurbituril is synthesized by condensation reaction between the disubstituted cucurbituril of the formula 1 and the silane compound of the formula 3.

As used herein, the silane compound is commercially available from Aldrich, TCI, etc., under the trade name such as 3-glycidoxypropyltrimethoxysilane, 3-aminopropyltriethoxysilane, and triethoxysilylpropylisocyanate.

Examples of the silane compound-bonded disubstituted cucurbituril include compounds represented by the following formulae 8 through 10:

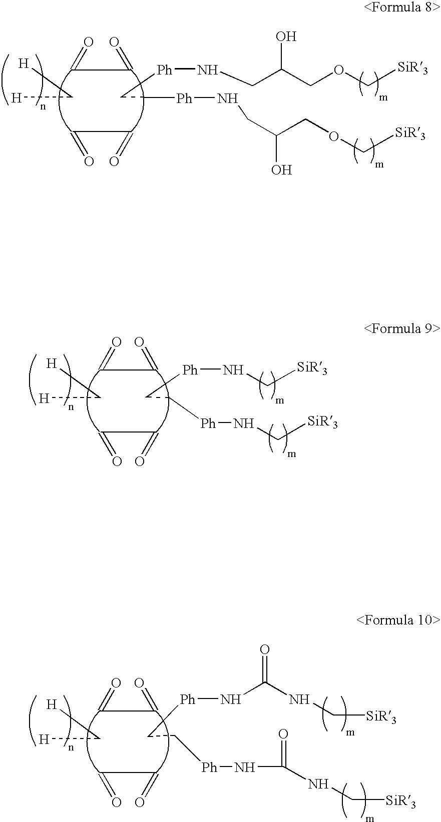

wherein n is an integer of 4-7, m is an integer of 3-10, R′ is selected from methoxy, ethoxy, chlorine, and bromine, and -Ph- is phenylene.

The compounds of the formulae 8-10 are synthesized by condensation reaction between diaminophenylcucurbituril of the formula 1 wherein each R is a phenyl group having an amino substituent at 3 (meta) or 4 (para) position and the silane compound of the formula 3. At this time, there are used silane compounds of the formula 3 wherein X is a glycidoxyalkyl group with an end epoxy moiety, an isothiocyanatoalkyl group, and a haloalkyl group, respectively, for syntheses of the compounds of the formulae 8, 9, and 10.

The condensation reaction between the diaminophenylcucurbituril of the formula 1 and the silane compound of the formula 3 is carried out in an organic solvent such as DMSO and DMF at a temperature range from 20 to 100° C.

The preparation of the disubstituted cucurbituril-bonded silica gel from the above-described silane compound-bonded disubstituted cucurbituril is as follows.

First, the diaminophenylcucurbituril-bonded silane compound of the formula 8, 9, or 10, as synthesized in the above, is dissolved in a solvent such as DMSO, DMF, benzene, toluene, and xylene. Then, the silica gel of the formula 2a is added thereto and heated at a high temperature of 60-130° C. for 10-60 hours to give the disubstituted cucurbituril-bonded silica gel of the formula 4, 6, or 7.

As described above, the disubstituted cucurbituril-bonded silica gels of the formulae 4 through 7 can be prepared by the following two methods.

A first method involves a condensation reaction between diaminophenylcucurbituril of the formula 1 wherein each R is a phenyl group having an amino substituent at 3 (meta) or 4 (para) position and the modified silica gel of the formula 2.

A second method involves a condensation reaction between diaminophenylcucurbituril of the formula 1 wherein each R is a phenyl group having an amino substituent at 3 (meta) or 4 (para) position and the silane compound of the formula 3 followed by reaction with the silica gel of the formula 2a.

The present invention also provides a silica monolithic column tube using the above-described silane compound-bonded disubstituted cucurbituril.

The silica monolithic column tube can be fabricated according to a known method (U.S. Pat. No. 6,638,885). An exemplary fabrication method of the silica monolithic column tube is as follows.

First, tetraalkyloxysilane (e.g., tetramethyloxysilane, tetraethyloxysilane, etc.) is placed in an acetic acid solution, which is used as a surfactant, containing polyethyleneglycol with a weight average molecular weight of 1,000 to 100,000, in particular 10,000, and urea, and stirred at 0-10° C., in particular 0° C., for 30 minutes to 1 hour. Then, the reaction solution is filled in a fused-silica capillary tube, aged at 20-50° C., in particular 40° C. for 2-20 hours, incubated at 80-200° C., in particular 120° C., for 3 hours, and washed with methanol and water. The resultant capillary tube is dried and thermally treated at 240-400° C., in particular 330° C. to burn up an organic substance.

After the organic substance treatment is completed as described above, both end spaces of the capillary tube are cut to make a silica monolithic capillary tube.

The silane compound-bonded disubstituted cucurbituril of the formula 8, 9, or 10 is dissolved in a solvent such as DMSO, DMF, methanol, toluene, benzene, and xylene, and then, the resultant solution is allowed to flow down through the above-described silica monolithic capillary tube at 50-70° C. A silica monolithic column tube thus fabricated is a capillary tube packed with the compound represented by the formula 4, 6, or 7.

The present invention also provides a column packing material using the above-described disubstituted cucurbituril-bonded silica gel and its use.

An exemplary preparation method of a column packing material for column chromatography using one of various disubstituted cucurbituril-bonded silica gels of the present invention is as follows.

To use the disubstituted cucurbituril-bonded silica gels of the formulae 4 through 7 as packing materials for column chromatography, first, the disubstituted cucurbituril-bonded silica gel of the formula 4, 5, 6, or 7 is added to a solvent such as methanol and ethanol to make a slurry, which is then allowed to flow down through a steel tube, an end of which is sealed by a silica gel.

While the slurry flows down through the inside of the steel tube, the disubstituted cucurbituril-bonded silica gel contained in the slurry is closely packed in the steel tube. After the disubstituted cucurbituril-bonded silica gel is packed in the steel tube, the steel tube is several times washed with a solvent such as methanol, ethanol, and water, and then attached to a system for column chromatography such as HPLC (High Performance Liquid Chromatography) and GC (Gas Chromatography) according to a separation purpose, to be used as a packing material for column chromatography. At this time, the steel tube is appropriately selected according to a separation purpose and a system for column chromatography. Preferably, a steel tube of 50 μm-5 mm in diameter and 1-10 cm in length is used for a micro-column and a steel tube of 0.2-2 cm in diameter and 5-25 cm in length is used for a common column. The solvent used is allowed to flow down through the steel tube for about two hours. Preferably, the solvent is a mixture solvent to be used in compound separation and is allowed to flow down through the steel tube for about 2 hours or more before use.

The above-described disubstituted cucurbituril-bonded silica monolithic column tube or steel tube packed with the silica gel of one of the formulae 4-7 can be used as a stationary phase or a filter for column chromatography such as HPLC, GC, SFC (Supercritical Fluid Chromatography), CE (Capillary Electrophoresis), and CEC (Capillary Electrokinetic Chromatography).

As described above, a disubstituted cucurbituril of the present invention is non-covalently bonded with various compounds such as biologically important amino acids, proteins, nucleic acids, optically or non-optically active asymmetrical substances, drugs, ionic substances, amines, and gaseous compounds. Based on such a non-covalent bond, various column stationary phases linked with the disubstituted cucurbituril of the present invention can be used in separation and purification of these various compounds. Furthermore, the column stationary phases can be efficiently used as filters for separation of various living body substances such as alkylamines, arylamines, polypeptides, and neural substances, and removal of ecologically harmful water contaminants or air pollutants such as aromatic compounds, dyes, and heavy metal ions.

Hereinafter, the present invention will be described more specifically by Examples. However, the following Examples are provided only for illustrations and thus the present invention is not limited to or by them.

### SYNTHESIS EXAMPLE 1

### Synthesis of Silica Gel of Formula 2 where R₂ is 3-glycidoxypropyl Group

1 g of a silica gel was dried at 100° C. under reduced pressure for 12 hours and 20 mL of toluene was added thereto. 5 mL of 3-glycidoxypropyltrimethoxysilane was added to the mixed solution, refluxed for 50 hours, washed with toluene, methanol, acetone, and diethylether, and dried under reduced pressure, to give a modified silica gel of formula 2 where R₂ is a 3-glycidoxypropyl group.
¹³C CP MAS NMR(75 MHz): δ=75.0, 66.5, 61.9, 56.5, 10.1.

### SYNTHESIS EXAMPLE 2

### Synthesis of Silica Gel of Formula 4 where n=5 and m=3

2.5 g of diaminophenylcucurbit[6]uril of formula 1 where n=5 and R=3-aminopheyl group was dissolved in 110 mL of dimethylsulfoxide. Then, 1 g of a modified silica gel of formula 2 where R₂ is a 3-glycidoxypropyl group was added thereto and stirred at 80° C. for 50 hours. After the reaction terminated, the reaction solution was washed with dimethylsulfoxide, water, acetone, methanol, and diethylether and dried under reduced pressure to give a disubstituted cucurbituril-bonded silica gel of formula 4 where n=5 and m=3.
¹³C CP MAS NMR(75 MHz): δ=158.4, 132.4, 123.2, 87.2, 73.1, 53.8, 32.3, 24.5, 11.7

### SYNTHESIS EXAMPLE 3

### Synthesis of Silane Compound of Formula 8 where n=5 and m=3

2.9 g of diaminophenylcucurbit[6]uril of formula 1 where n=5 and R=3-aminopheyl group was dissolved in 40 mL of dimethylsulfoxide. Then, 1.1 mL of 3-glycidoxypropyltrimethoxysilane was added thereto and stirred at 80° C. for 30 hours.

After the reaction terminated, a precipitate was removed by addition of acetone. Then, the resultant solution was washed with acetone and diethylether and dried to give a disubstituted cucurbituril-bonded silane compound of formula 8 where n=5 and m=3.
¹H NMR(500 MHz, DMSO-d6): δ 0.71(t, J=15 Hz), 1.84 (m), 3.25 (m), 3.45 (s), 3.60 (m), 3.97 (m), 4.02 (m), 4.43 (m), 5.27 (d, J=10.0), 5.56 (d, J=10.0 Hz), 5.70 (m), 5.80 (m), 5.97 (t, J=15.0 Hz), 6.26 (s), 6.39 (m), 6.62 (m), 7.04 (m).

### SYNTHESIS EXAMPLE 4

### Synthesis of silica gel of formula 4 where n=5 and m=3

1 g of a silica gel was dried at 100° C. under reduced pressure for 12 hours and then 20 mL of dimethylsulfoxide was added thereto. 400 mg of a disubstituted cucurbituril-bonded silane compound of formula 8 where n=5 and m=3 was added thereto and stirred at 80° C. for 3 days.

After the reaction terminated, the reaction solution was several times washed with dimethylsulfoxide, water, acetone, methanol, and diethylether and dried under reduced pressure to give a disubstituted cucurbituril-bonded silica gel of formula 4 where n=5 and m=3.
¹³C CP MAS NMR (75 MHz): δ=157.3, 131.2, 122.2, 89.2, 74.3, 54.8, 32.6, 26.2, 10.7

### SYNTHESIS EXAMPLE 5

### Synthesis of silica gel of formula 7 where n=5 and m=3

2.4 g of diaminophenylcucurbit[6]uril of formula 1 where n=5 and R=3-aminopheyl group was dissolved in 80 mL of dimethylsulfoxide. Then, 1 g of a silica gel of formula 2 where R₂ is a 3-isocyanatopropyl group was added thereto and stirred at 80° C. for 50 hours.

After the reaction terminated, the reaction solution was washed with dimethylsulfoxide, water, acetone, methanol, and diethylether and dried under reduced pressure to give a disubstituted cucurbituril-bonded silica gel of formula 7 where n=5 and m=3.
¹³C CP MAS NMR(75 MHz): δ=158.7, 152.0, 132.5, 126.8, 118.3, 45.3, 23.9, 5.9.

### SYNTHESIS EXAMPLE 6

### Synthesis of silica gel of formula 5 where n=5 and m=3

1 g of a silica gel of formula 2 where R₂ is a 3-aminopropyl group was added to 80 mL of dimethylsulfoxide. Then, 260 mL of 1,4-phenylene diisothiocyanate was added thereto and stirred at 100° C. for 24 hours. 1.5 g of diaminophenylcucurbit[6]uril of formula 1 where R is a 3-aminopheyl group was added thereto and stirred at 100° C. for 60 hours.

After the reaction terminated, there was obtained a disubstituted cucurbituril-bonded silica gel of formula 5 where n=5 and m=3.
¹³C CP MAS NMR (75 MHz): δ=182.8, 174.7, 158.5, 129.4, 88.6, 72.7, 53.3, 44.2, 31.8, 23.4, 11.7.

### EXAMPLE 1

### Fabrication of Disubstituted Cucurbituril-Bonded Silica Monolithic Column Tube

4 mL of tetramethoxysilane was added to 10 mL of a 0.01 M acetic acid solution containing 0.88 g of polyethyleneglycol with a molecular weight of 10,000 and 0.9 g of urea at 0° C. for 45 minutes. A fused-silica capillary tube activated with a 1 M sodium hydroxide solution at 40° C. for 3 hours was filled with the previously prepared mixed solution and incubated at 40° C. for 2 hours to form a gel. The resultant capillary tube was aged for 20 hours, incubated at 120° C. for 3 hours, washed with water and methanol, and thermally treated at 330° C. for 25 hours. Incompletely packed portions of both sides of the capillary tube were cut to give a silica monolithic capillary tube with a length of 15 cm. A solution obtained by dissolving 1.1 g of a disubstituted cucurbituril-bonded silane compound of formula 8 where n=5 and m=3 in 100 mL of dimethylsulfoxide was allowed to continuously flow down through the silica monolithic capillary tube at 60° C. under a pressure of 50 mbar for 3 hours. Then, the capillary tube was several times washed with dimethylsulfoxide, water, and methanol and dried to give a disubstituted cucurbituril-bonded silica monolithic column tube.

### EXAMPLE 2

### Fabrication of Column Tube Using Disubstituted Cucurbituril-Bonded Silica Gel

An end of a steel tube with an inner diameter of 0.6 cm and length of 10 cm was sealed with a silica gel. A slurry obtained by adding 1 g of a silica gel of formula 4 where n=5 and m=3 to methanol was allowed to flow down through the steel tube by a slurry packing method to obtain a steel tube packed with a disubstituted cucurbituril-bonded silica gel of formula 4. The steel tube was washed with a large amount of methanol under a pressure of 2,000 psi for 3 hours, dried, and attached to a HPLC machine, to be used as a column tube for HPLC.

### EXAMPLE 4

### Separation of Amino Acids Using Disubstituted Cucurbituril-Bonded Silica Gel Stationary Phase

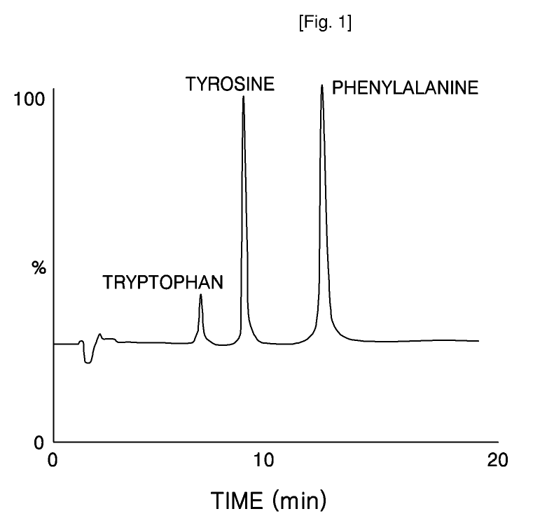

Separation of three amino acids, tryptophan, tyrosine, and phenylalanine was performed using a HPLC machine to which a column tube packed with a disubstituted cucurbituril-bonded silica gel of formula 4 where n=5 and m=3 was attached. A mixed solution of the three amino acids was injected in acetonitrile. At this time, an eluent (acetonitrile:water=40:60 v/v) was allowed to flow at a flow rate of 0.5 mL/min and the separation of the three amino acids was observed by a UV detector (see FIG. 1). In FIG. 1, 1 is tryptophan, 2 is tyrosine, and 3 is phenylalanine, and a flow rate is 0.5 mL/min.

While the above Synthesis Examples have been particularly shown and described in terms of only specific bonds between cucurbituril and a silica gel, it will be understood by those of ordinary skill in the art that synthesis of a silica gel linked with cucurbituril is possible by various types of bonds.

Unlike a simple mixture, a silica gel covalently bonded with disubstituted cucurbituril according to the present invention can be covalently linked to a solid support, thereby enabling recycling of a solid support containing a constant content of cucurbituril. The silica gel is not dissolved in a stationary phase, and thus easily separated, which ensures a more efficient use of the silica gel. Furthermore, the silica gel has selective separation capability for various test samples because it can have various types of covalent bond lengths and various types of functional groups which participate in the covalent bonds. Therefore, a solid phase such as a disubstituted cucurbituril-bonded silica gel can be used in various applications such as various types of stationary column packing materials for separation and purification technologies and filters for removal of various contaminants.

What is claimed is:

1. A disubstituted cucurbituril-bonded silica gel in which (i) a disubstituted cucurbituril represented by the following formula 1 is bonded to a modified silica gel represented by the following formula 2 or (ii) a silane compound represented by the following formula 3 bonded to the disubstituted cucurbituril represented by the following formula 1 is bonded to an unmodified silica gel represented by the following formula 2a: 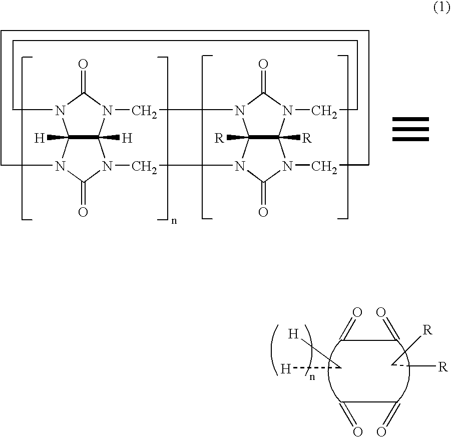
wherein,
n is an integer of 4-7;
Rs are the same or different and each represents a substituted or unsubstituted alkenyl group of C2-C30, a substituted or unsubstituted alkynyl group of C2-C30, a substituted or unsubstituted alkylcarboxyl group of C1-C30, a substituted or unsubstituted hydroxyalkyl group of C1-C30, a substituted or unsubstituted alkoxy group of C1-C30, a substituted or unsubstituted nitroalkyl group of C1-C30, a substituted or unsubstituted aminoalkyl group of C1-C30, a substituted or unsubstituted aryl group of C6-C30, or a substituted or unsubstituted heteroaryl group of C2-C30, 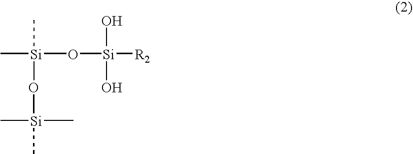
wherein R₂ represents a halogenated alkyl group of C1-C10, a mercaptoalkyl group of C1-C10, an aminoalkyl group of C1-C10, an epoxyalkyloxyalkyl group of C2-C10, an isocyanatoalkyl group of C2-C10, or an isothiocyanatoalkyl group of C2-C10, 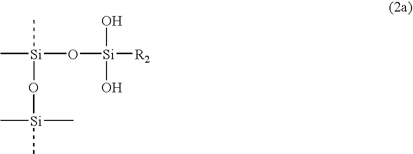
wherein R₂ is a hydroxy group, and
XSiR′₃ (3)
wherein X represents a halogenated alkyl group of C1-C10, an aminoalkyl group of C1-C10, an epoxyalkyloxyalkyl group of C2-C10, an isocyanatoalkyl group of C2-C10, or an isothiocyanatoalkyl group of C2-C10; and R′s are the same or different and each represents a hydrogen, a halogen atom, an aryl group, an alkyl group of C1-C20, a halogenated alkyl group of C1-C20, or an alkyloxy group of C1-C20.

2. The disubstituted cucurbituril-bonded silica gel of claim 1, which is one of compounds represented by the following formulae 4 through 7: 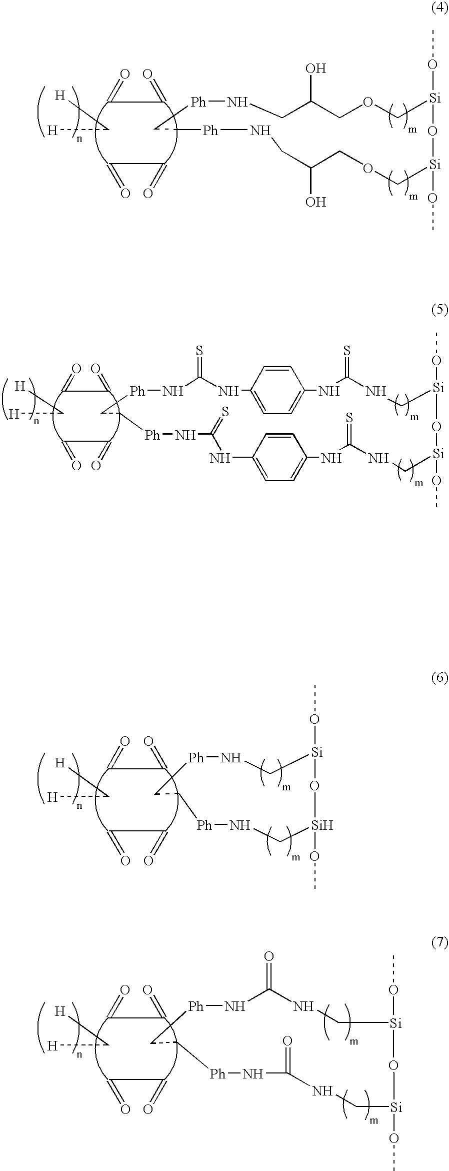
wherein n is an integer of 4-7, m is an integer of 1-10, and -Ph- is phenylene.

3. A silica monolithic column tube covalently bonded with a disubstituted cucurbituril obtained by allowing a disubstituted cucurbituril-bonded silane compound obtained by bonding a disubstituted cucurbituril of the following formula 1 to a silane compound of the following formula 3 to pass through a silica monolithic capillary tube: 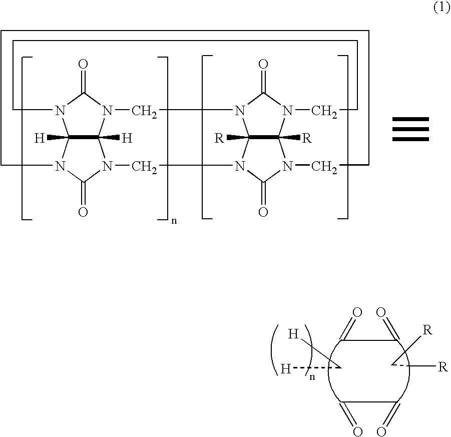
wherein,
n is an integer of 4-7;
Rs are the same or different and each represents a substituted or unsubstituted alkenyl group of C2-C30, a substituted or unsubstituted alkynyl group of C2-C30, a substituted or unsubstituted alkylcarboxyl group of C1-C30, a substituted or unsubstituted hydroxyalkyl group of C1-C30, a substituted or unsubstituted alkoxy group of C1-C30, a substituted or unsubstituted nitroalkyl group of C1-C30, a substituted or unsubstituted aminoalkyl group of C1-C30, a substituted or unsubstituted aryl group of C6-C30, or a substituted or unsubstituted heteroaryl group of C2-C30, and
XSiR′₃ (3)
wherein X represents a halogenated alkyl group of C1-C10, an aminoalkyl group of C1-C10, an epoxyalkyloxyalkyl group of C2-C10, an isocyanatoalkyl group of C2-C10, or an isothiocyanatoalkyl group of C2-C10; and
R′s are the same or different and each represents a hydrogen, a halogen atom, an allyl group, an alkyl group of C1-C20, a halogenated alkyl group of C1-C20, or an alkyloxy group of C1-C20.

4. The silica monolithic column tube of claim 3, wherein the disubstituted cucurbituril-bonded silane compound is selected from compounds represented by the following formulae 8 through 10: 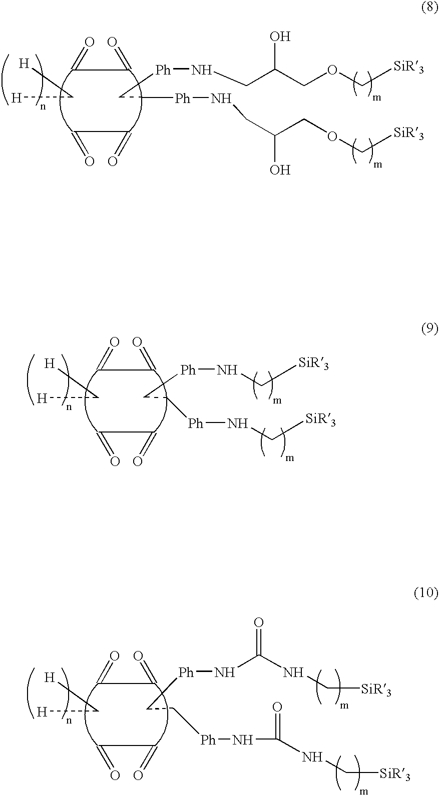
wherein n is an integer of 4-7, m is an integer of 1-10, R′ is selected from methoxy, ethoxy, chlorine, and bromine, and -Ph- is phenylene.

5. The silica monolithic column tube of claim 3, which is packed with a compound represented by the following formula 4, 6, or 7: 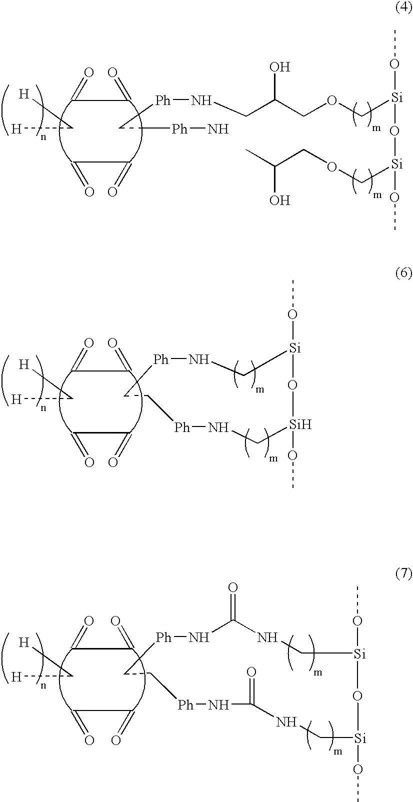
wherein n is an integer of 4-7, m is an integer of 1-10, and -Ph- is phenylene.

6. A method of preparing the disubstituted cucurbituril-bonded silica gel of claim 1, which comprises reacting a disubstituted cucurbituril represented by the following formula 1 with a modified silica gel represented by the following formula 2: 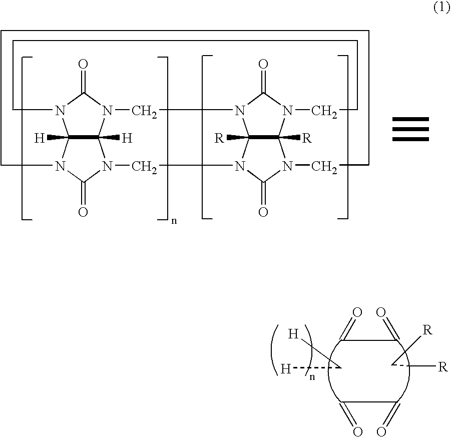
wherein,
n is an integer of 4-7;
Rs are the same or different and each represents a substituted or unsubstituted alkenyl group of C2-C30, a substituted or unsubstituted alkynyl group of C2-C30, a substituted or unsubstituted alkylcarboxyl group of C1-C30, a substituted or unsubstituted hydroxyalkyl group of C1-C30, a substituted or unsubstituted alkoxy group of C1-C30, a substituted or unsubstituted nitroalkyl group of C1-C30, a substituted or unsubstituted aminoalkyl group of C1-C30, a substituted or unsubstituted aryl group of C6-C30, or a substituted or unsubstituted heteroaryl group of C2-C30, and 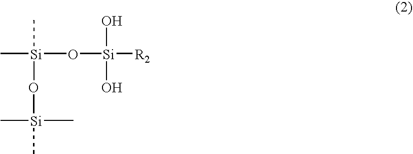
wherein R₂ represents a halogenated alkyl group of C1-C10, a mercaptoalkyl group of C1-C10, an aminoalkyl group of C1-C10, an epoxyalkyloxyalkyl group of C2-C10, an isocyanatoalkyl group of C2-C10, or an isothiocyanatoalkyl group of C2-C10.

7. A method of preparing the disubstituted cucurbituril-bonded silica gel of claim 1, which comprises reacting a disubstituted cucurbituril represented by the following formula 1 with a silane compound represented by the following formula 3 followed by reacting with an unmodified silica gel represented by the following formula 2a: 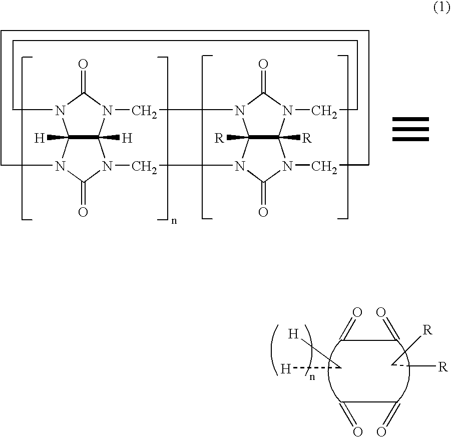
wherein,
n is an integer of 4-7;
Rs are the same or different and each represents a substituted or unsubstituted alkenyl group of C2-C30, a substituted or unsubstituted alkynyl group of C2-C30, a substituted or unsubstituted alkylcarboxyl group of C1-C30, a substituted or unsubstituted hydroxyalkyl group of C1-C30, a substituted or unsubstituted alkoxy group of C1-C30, a substituted or unsubstituted nitroalkyl group of C1-C30, a substituted or unsubstituted aminoalkyl group of C1-C30, a substituted or unsubstituted aryl group of C6-C30, or a substituted or unsubstituted heteroaryl group of C2-C30, 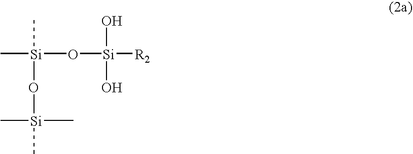
wherein R₂ is a hydroxy group, and
XSiR′₃ (3)
wherein X represents a halogenated alkyl group of C1-C10, an aminoalkyl group of C1-C10, an epoxyalkyloxyalkyl group of C2-C10, an isocyanatoalkyl group of C2-C10, or an isothiocyanatoalkyl group of C2-C10; and R′s are the same or different and each represents a hydrogen, a halogen atom, an allyl group, an alkyl group of C1-C20, a halogenated alkyl group of C1-C20, or an alkyloxy group of C1-C20.

8. A method of fabricating the disubstituted cucurbituril-bonded silica monolithic column tube of any one of claim 3, which comprises:
reacting a disubstituted cucurbituril represented by the following formula 1 with a silane compound represented by the following formula 3 to obtain a disubstituted cucurbituril-bonded silane compound; and
allowing the disubstituted cucurbituril-bonded silane compound to pass through a silica monolithic capillary tube to obtain a disubstituted cucurbituril-covalently bonded silica monolithic column tube: 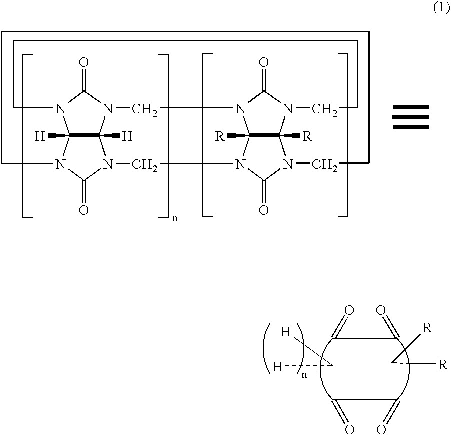
wherein,
n is an integer of 4-7;
Rs are the same or different and each represents a substituted or unsubstituted alkenyl group of C2-C30, a substituted or unsubstituted alkynyl group of C2-C30, a substituted or unsubstituted alkylcarboxyl group of C1-C30, a substituted or unsubstituted hydroxyalkyl group of C1-C30, a substituted or unsubstituted alkoxy group of C1-C30, a substituted or unsubstituted nitroalkyl group of C1-C30, a substituted or unsubstituted aminoalkyl group of C1-C30, a substituted or unsubstituted aryl group of C6-C30, or a substituted or unsubstituted heteroaryl group of C2-C30, and
XSiR′₃ (3)
wherein X represents a halogenated alkyl group of C1-C10, an aminoalkyl group of C1-C10, an epoxyalkyloxyalkyl group of C2-C10, an isocyanatoalkyl group of C2-C10, or an isothiocyanatoalkyl group of C2-C10; and R′s are the same or different and each represents a hydrogen, a halogen atom, an allyl group, an alkyl group of C1-C20, a halogenated alkyl group of C1-C20, or an alkyloxy group of C1-C20.

9. A column packing material using the disubstituted cucurbituril-bonded silica gel of claim 1.

10. A filter using the disubstituted cucurbituril-bonded silica gel of claim 1.

11. A use of the silica monolithic column tube of claim 3 in separation of alkylamines, arylamines, polypeptides, or neural substances.

12. A use of the column packing material of claim 9 in separation of ionized alkaline or transition metal compounds, hydrophilic amino acids, alkaloids, proteins, nucleic acids, optically or non-optically active asymmetrical substances, drugs, ionic substances, amines, or gaseous compounds.

13. A use of the filter of claim 10 in separation of ionized alkaline or transition metal compounds, hydrophilic amino acids, alkaloids, proteins, nucleic acids, optically or non-optically active asymmetrical substances, drugs, ionic substances, amines, or gaseous compounds.
